(19) United States
(12) Patent Application Publication
(10) Pub. No.: US 2003/0170609 A1
(43) Pub. Date: Sep. 11, 2003

(54) Method for selecting particles

(76) Inventor: Rudolf Rigler, St-Sulpice (CH)

Correspondence Address: FULBRIGHT & JAWORSKI, LLP, 666 FIFTH AVE, NEW YORK, NY, 10103-3198, US

(21) Appl. No.: 10/312,092

(22) PCT Filed: Jun. 25, 2001
(86) PCT No.: PCT/EP01/07190

(30) Foreign Application Priority Data
Jun. 26, 2000 (DE) 100 31 028.1

(51) Int. Cl. C12Q 1/00; C12Q 1/70; C12Q 1/68; G01N 33/53

(52) U.S. Cl. 435/4; 435/5; 435/6; 435/7.1

(57) ABSTRACT

The invention relates to a method for selecting particles having a predetermined property from a population of a multiplicity of different particles and to a device suitable for carrying out said method.

The invention relates to a method for selecting particles having a predetermined property from a population of a multiplicity of different particles and to a device suitable for carrying out said method.

In order to identity new ligands for diagnostic, biomedical and pharmaceutical applications, it is possible to use combinatorial libraries comprising a population of a multiplicity of particles, for example phages, cells, ribosomes, etc., the individual particles in each case presenting different ligands (see, for example, WO 90/02809; WO 92/15677; WO 92/15679; WO 92/06204; WO 92/06176; WO 90/19162; WO 98/35232; WO 99/06839 and WO 99/5428). Ligands having a predetermined property are usually identified by screening the library to be investigated, in which process a labeled target molecule is contacted with the individual particles of said library and the occurrence of binding between said target molecule and a particular particle of said library or the ligand presented by said particle, respectively, is determined. Subsequently, the particle having the predetermined property needs to be identified. However, previous selection and identification methods, for example the “Penning” or “Selex” methods, are relatively inefficient so that it is often not possible to find a particular particle with desired properties in the library, although it is present therein.

European patent 0 679 251 describes direct detection of individual analyte molecules in the form of the fluorescence correlation spectroscopy (FCS) method. It is possible to detect by means of FCS a single molecule or just a few molecules labeled with fluorescent dyes in a small measuring volume of, for example, <10⁻¹⁴ l.

The measuring principle of FCS is based on exposing a small volume element of the sample fluid to a strong excitation light, for example of a laser, so that only those fluorescent molecules are excited which are present in said measuring volume. The fluorescence light emitted from said volume element is then projected onto a detector, for example a photo-multiplier. A molecule in the volume element disappears from the latter again according to its characteristic rate of diffusion after an average period of time which is, however, characteristic for the molecule in question and can then no longer be observed.

If the luminescence of one and the same molecule is then excited repeatedly during its average residence time in the measuring volume, it is possible to record a multiplicity of signals from said molecule.

Eigen and Rigler (Proc. Natl. Acad. Sci. USA 91 (1994), 5740-5747) and Rigler (J. Biotech. 41 (1995), 177-186) describe the application of fluorescence correlation spectroscopy to sorting and identifying individual molecules. The use of a quadrupole trap and electric field gradients in conjunction with single-photon detectors for identifying single molecules is proposed. Although this method is substantially more efficient than the classical selection procedures, the selection of single molecules requires the use of extremely low particle concentrations and is time-consuming. Therefore, there is a need for improving sensitivity and efficiency for selecting particles.

This object is achieved by a method for selecting a particle having a predetermined property from a population of a multiplicity of different particles, comprising the following steps:

(a) providing a population of different particles,

(b) labeling particles which have a said predetermined property,

(c) passing the particles in a microchannel through a detection element which can distinguish between labeled and unlabeled particles,

(d) removing labeled particles, and

(e) repeating at least once the steps (c) and (d), reducing the concentration of said particles in a subsequent cycle compared to a preceding cycle.

The method of the invention makes it possible to select individual particles from very large particle populations which comprise, for example, more than 10⁸ or even 10¹² or more different particles. The particles may be cells, cell surface parts, cell organelles, for example ribosomes, viruses such as, for example, bacteriophages, e.g. filamentous phages or plasmids packaged in phage envelopes (phagemids), nucleic acids such as genes or cDNA molecules, proteins such as, for example, enzymes or receptors or low molecular weight substances. The particles are preferably elements of a combinatorial library, for example a library of genetic packages such as phages, cells, spores or ribosomes, which on their surface present peptide structures, for example linear or circular peptides, or proteins such as antibodies, preferably fused to surface proteins, for example surface proteins of filamentous phages.

The method of the invention makes it possible to select efficiently a particle having a predetermined property from a multiplicity of different particles. The term “predetermined property” in accordance with the present invention means preferably the ability to bind to a target substance. Binding of the particle to the target substance may comprise ligand-receptor binding, enzyme-substrate binding, antibody-antigen binding, nucleic acid hybridization, sugar-lectin binding or another biological interaction with high affinity. On the other hand, the predetermined property of said particle may also comprise preventing a biological interaction, for example binding to a target substance.

The particle having the predetermined property is selected by incubating the particle population preferably with a target substance carrying a detectable label, the incubation conditions being chosen such that the particle having the predetermined property binds to a labeling group and can thus be removed from other particles. Suitable labeling groups are in particular nonradioactive labeling groups and, particularly preferably, labeling groups detectable by optical methods, such as, for example, dyes and, in particular, fluorescent labeling groups. Examples of suitable fluorescent labeling groups are rhodamine, Texas Red, phycoerythrin, fluorescein and other fluorescent dyes common in diagnostic methods or selection methods.

The labeled target substance is specific for the particle to be identified, i.e. the target substance binds to the particle having the predetermined property with sufficiently high affinity and selectivity under the test conditions in order to make selection possible.

The predetermined property of the particle to be selected may, where appropriate, also be a biological activity, for example an enzymic activity. In this case, it is possible to incubate the particles with a chromogenic or fluorescent enzyme substrate and encapsulate them in vesicles, for example lipid vesicles such as liposomes. If a particle, for example a phage or a ribosome, presents an active enzyme molecule on its surface, the substrate is converted inside the vesicle, resulting in a colored or fluorescent product which can be detected.

In order to distinguish labeled particles, i.e. particles having the predetermined property, and unlabeled particles, i.e. particles without said predetermined property, said particles are passed in a microchannel through a detection element. Passing through the microchannel is preferably carried out using a hydrodynamic flow, for example by means of suction or pumping action. However, the flow may also be an electroosmotic flow which is generated by an electric field gradient. A combination of hydrodynamic flow and field gradient is also possible. The flow through the microchannel preferably has a parabolic flow profile, i.e. the flow rate is highest in the center of the microchannel and decreases down to a minimum rate toward the edges in a parabolic function. The flow rate through the microchannel, at maximum, is preferably in the range from 1 to 50 mm/s, particularly preferably in the range from 5 to 10 mm/s. The microchannel diameter is preferably in the range from 1 to 100 μm, particularly preferably from 10 to 50 μm. The measurement is preferably carried out in a linear microchannel which essentially has a constant diameter.

A labeled particle may be identified by means of any measuring method, for example using space- and/or time-resolved fluorescence spectroscopy which is capable of recording very small signals of labeling groups, in particular fluorescent signals down to the single-photon counting, in a very small volume element as is found in a microchannel. In this connection, it is important that the signals originating from labeled particles are distinctly different from those caused by the labeled particles.

The detection may be carried out, for example, by means of fluorescence correlation spectroscopy in which a very small confocal volume element, for example 0.1 to 20×10⁻¹⁵ l of the sample fluid flowing through the microchannel, is exposed to an excitation light of a laser, which causes the receptors present in this measuring volume to emit fluorescence light, the fluorescence light emitted from said measuring volume being measured by means of a photodetector, and a correlation between the time-dependent change in the emission measured and the relative flow rate of the molecules involved being made so that it is possible, at an appropriately high dilution, to identify individual molecules in said measuring volume. For details of carrying out the method and of the apparatuses used for detection, reference is made to the disclosure of European patent 0 679 251.

Alternatively, detection may also be carried out via a time-resolved decay measurement, so-called time gating, as described, for example, by Rigler et al., “Picosecond Single Photon Fluorescence Spectroscopy of Nucleic Acids”, in: “Ultrafast Phenomenes”, D. H. Auston, Ed., Springer 1984. In this case, the fluorescent molecules are excited in a measuring volume and, subsequently, preferably at a time interval of ≧100 ps, a detection interval on the photodetector is opened. In this way it is possible to keep background signals generated by Raman effects sufficiently low so as to make possible an essentially interference-free detection.

The device for detecting fluorescently labeled particles in the sample fluid flowing through the microchannel particularly preferably comprises a laser as a fluorescence excitation light source for the molecules, an optical arrangement for directing and focusing laser light of the laser to a focal region of the microchannel and for confocally projecting the focal region to a photodetector arrangement for recording fluorescence light which has been emitted in the focal region by one or, where appropriate, more optically excited molecules, the optical arrangement having in the laser beam path a diffraction element or a phase-modulating element which, where appropriate in combination with one or more optical imaging elements, is arranged in order to generate from the laser beam of the laser a diffraction pattern in the form of a linear or two dimensional array of focal regions in the microchannel, said optical arrangement being arranged in order to project each focal region confocally for fluorescence detection by the photodetector arrangement. Alternatively, the detection device may have two walls which mark the boundary of the microchannel on opposite sides and one of which has an array of preferably integrated laser elements emitting into the microchannel as fluorescence excitation light sources and the other one of which has an array of preferably integrated photodetector elements, arranged in each case opposite the laser elements, as fluorescence light detectors, said laser elements being preferably quantum well laser elements and said photodetector elements being preferably avalanche diodes. Such devices are described, for example, in DE 100 23 423.2.

The labeled particles identified by the detection element are removed from unlabeled particles, and this may be carried out using a sorting procedure as described in Holm et al (Analytical Methods and Instrumentation, Special Issue μTAS 96, 85-87), Eigen and Rigler (Proc. Natl. Acad. Sci. USA 91 (1994), 5740-5747) or Rigler (J. Biotech 41 (1995), 177-186). The sorting procedure is preferably automated, with labeled and unlabeled particles being directed into different branches of the microchannel. The sorting procedure is preferably controlled by switching a valve which is either external or integrated into the microstructure, after detecting a labeled particle in the detection element, so that the labeled particle is directed into the microchannel branch provided therefor and then switching said valve again so that unlabeled particles are directed into the other microchannel branch.

The method of the invention is a cascade process which comprises repeating, where appropriate several times, the detection and removal steps. While the procedure for selecting single molecules, which is known from the prior art, can be carried out reliably only at extremely high dilutions and thus in very large volumes, the concentration of the particles passed through the detection device is set at a sufficiently high level in the method of the invention so that it is possible to maintain a small total sample fluid volume which is to be studied and which contains the entire particle population. The particle concentration used for the first selection cycle is preferably from 10⁸ to 10¹⁴ per 100 μl of sample volume and particularly preferably 10¹⁰ to 10¹² particles per 100 μl of sample volume. Although it is accepted that, under these conditions, in addition to the labeled particle also a number of other, negative particles, usually 10² to 10³ particles, are initially classified as positive, it is nevertheless possible, by means of subsequent selection cycles which are carried out in each case with a reduced concentration compared to a preceding cycle, to finally isolate individual particles which have the predetermined properties. The reduction in particle concentration is preferably chosen so as to be able to identify in a further selection step a positive particle unambiguously. It is possible, for example, to reduce the particle concentration per cycle by at least a factor of 10⁴, preferably by a factor of 10⁶ to 10⁸ and particularly preferably by approximately a factor of 10⁷. In this connection, the sample volume is generally not substantially increased, since the first selection cycle achieved a significant reduction in the number of particles. It is possible, where appropriate, to carry out one or more further cycles after the second selection cycle.

Furthermore, the method of the invention preferably comprises identifying or/and characterizing the particles found which have the predetermined property. This step may comprise, for example, an amplification, for example, in the case of cells and viruses, a propagation or, in the case of nucleic acids, an amplification reaction such as PCR, or sequencing. The identified or characterized particle or the characteristic determinant thereof, for example a protein presented on the surface, may then be used according to its particular intended purpose or as a basis for preparing another combinatorial library, for example by mutagenesis.

In a preferred embodiment of the method of the invention, a preselective affinity procedure is carried out after labeling the particles, but prior to introducing said particles into the detection device. To this end, labeling, for example treatment of the particle population with a labeled binding molecule, is followed by a further treatment step using unlabeled binding molecules, so that in particles which have bound the labeled binding molecule only weakly the unlabeled binding molecule can replace the labeled binding molecule by means of dissociation. These particles which are capable of weak binding and which are thus unwanted, are in this case not recognized as positive in the removal procedure from the outset and are therefore eliminated. It is possible to adjust the “stringency” of the preselective affinity process by adjusting the conditions for the treatment of labeled particles with unlabeled binding molecules. Increasing the incubation time, the temperature and the concentration of unlabeled binding molecules leads to an increase in stringency.

If the predetermined property of the particle consists of selective binding to a target substance but, if possible, not to a substance closely related to said target substance, an incubation with the closely related substance may be carried out prior to or/and after labeling of the target substance, so that particles which have an affinity for the closely related substance are not recorded in the removal procedure from the outset.

The invention further relates to a device for selecting a particle having a predetermined property from a population comprising a multiplicity of different particles, comprising:

(a) an optically transparent microchannel,

(b) means for introducing particles into said microchannel,

(c) means for detecting a label on a particle passed through said microchannel,

(d) means for removing a labeled particle from unlabeled particles,

which device is characterized in that the means (c) and (d) are designed in such a way that they provide for repeating at least once the detection/removal procedure.

Furthermore, the device preferably comprises automated manipulation devices, heating or cooling equipment such as Peltier elements, reservoirs and, where appropriate, supply lines for sample fluid and reagents and also electronic devices for evaluation.

The device is particularly suitable for carrying out the method of the invention.

The invention is furthermore intended to be illustrated by the following figures in which:

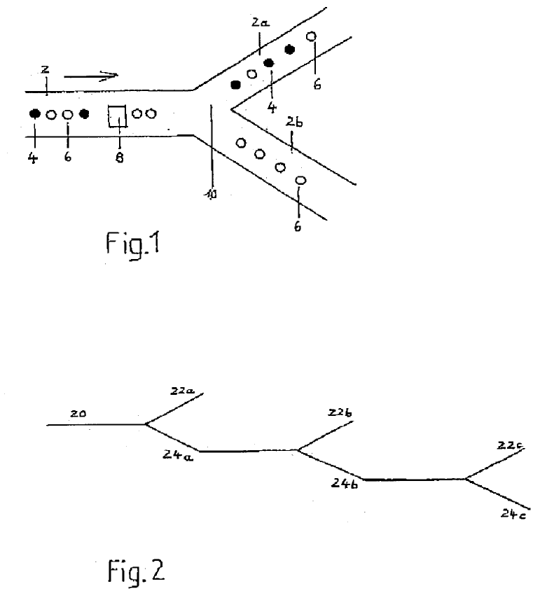

FIG. 1 depicts a section of a device for carrying out the method of the invention. Labeled particles (4) and unlabeled particles (6) are transported via a microchannel (2) to a detection element (8). Detection of a labeled particle (4) by the detection element (8) leads to the activation of valves (not shown) which are operated at the branching site (10) of the microchannel so that the labeled particles (4) are directed into the branch (2a) and unlabeled particles are directed into the branch (2b). The particle concentration or/and the rate of flow through the microchannel is/are chosen in the method of the invention so high that unlabeled particles (6) also enter the branch (2a) provided for labeled particles. Finally, by repeating, where appropriate several times, the selection/removal procedure, only labeled particles are obtained.

FIG. 2 depicts the principle of the cascade-like selection/removal procedure. The particles passed through the microchannel (20) are, as shown in FIG. 1, fractionated at a first branching into a microchannel arm (24a) provided for the labeled, particles and a microchannel arm (22a) provided for the unlabeled particles. The particles passed through the channel arm (24a) are fractionated at another branching again into an arm (24b) provided for labeled particles and an arm (22b) provided for unlabeled particles. The particles streaming through the microchannel (24b) may, where appropriate, be fractionated still further into an arm (24c) and an arm (22c).

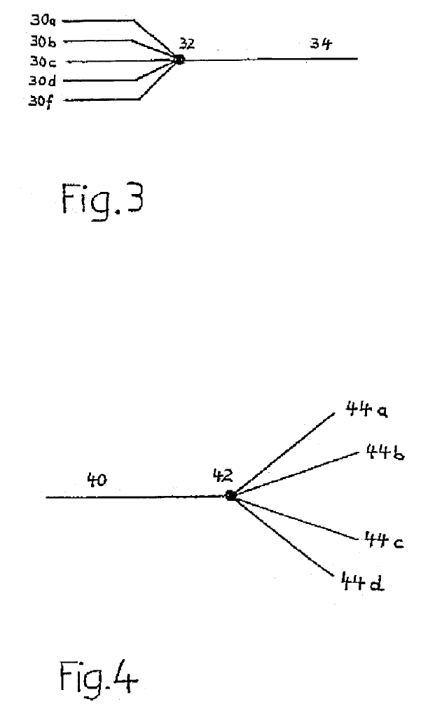

FIG. 3 depicts an embodiment of the device of the invention with multiple inlets. Particles from different sublibraries (30a, 30b, 30c, 30d, 30f) may be introduced at a switching valve (32) into a microchannel (34) and subjected there to the cascade selection/removal procedure depicted in FIG. 1 and FIG. 2.

FIG. 4 depicts an embodiment of the device of the invention with multiple outlets. The particles streaming through a microchannel (40) are fractionated at the branching site (42) into several arms (44a, 44b, 44c, 44d). Fractionation into more than two arms may be convenient, for example, when using a plurality of labeling groups, in order to separate particles with no labeling group, with in each case one labeling group or with a plurality of labeling groups from one another. Alternatively, they may also be separated on the basis of the intensity of the labeling by setting appropriate cutoff values on the detector.

1. A method for selecting a particle having a predetermined property from a population comprising a multiplicity of different particles, comprising the following steps:
(a) providing a population of different particles,
(b) labeling particles which have a said predetermined property,
(c) passing the particles in a microchannel through a detection element which can distinguish between labeled and unlabeled particles,
(d) removing labeled particles, and
(e) repeating at least once the steps (c) and (d), reducing the concentration of said particles in a subsequent cycle compared to a preceding cycle.

2. The method as claimed in claim 1, characterized in that the particles are selected from the group consisting of cells, cell surface parts, cell organelles, viruses, nucleic acids, proteins and low molecular weight substances.

3. The method as claimed in claim 1 or 2, characterized in that the population comprises a combinatorial library.

4. The method as claimed in claim 3, characterized in that the combinatorial library is selected from genetic packages such as phages, cells, spores or ribosomes.

5. The method as claimed in any of claims 1 to 4, characterized in that the population comprises more than 10⁸ different particles.

6. The method as claimed in claim 5, characterized in that the population comprises more than 10¹² different particles.

7. The method as claimed in any of claims 1 to 6, characterized in that the labeling comprises incubating the particles with a target substance carrying a detectable label.

8. The method as claimed in claim 7, characterized in that the label used is a fluorescent labeling group.

9. The method as claimed in any of claims 1 to 8, characterized in that the particles are passed through a microchannel of from 1 to 100 μm in diameter.

10. The method as claimed in any of claims 1 to 9, characterized in that the particles are passed through the microchannel by means of a hydrodynamic flow.

11. The method as claimed in any of claims 1 to 10, characterized in that the labeled particles are detected by fluorescence correlation spectroscopy.

12. The method as claimed in any of claims 1 to 10, characterized in that the labeled particles are detected by means of a time-resolved fluorescence decay measurement.

13. The method as claimed in any of claims 1 to 12, characterized in that the removing comprises directing the labeled particles and the unlabeled particles into different branches of the microchannel.

14. The method as claimed in any of the preceding claims, characterized in that the concentration in the first selection cycle of the particles passed through the microchannel is in the range from 10⁸ to 10¹⁴ per 100 μl of sample volume.

15. The method as claimed in any of the preceding claims, characterized in that the particle concentration is reduced by at least a factor of 10⁴ in a subsequent process cycle.

16. The method as claimed in any of the preceding claims, furthermore comprising identifying or/and characterizing a particle having the predetermined property.

17. The method as claimed in any of the preceding claims, furthermore comprising a preselective affinity step in which the labeled particles are exposed to conditions under which relatively weakly labeled particles lose their label.

18. The method as claimed in claim 17, characterized in that the labeling is followed by incubation with an unlabeled target substance.

19. A device for selecting a particle having a predetermined property from a population comprising a multiplicity of different particles, comprising:
(a) an optically transparent microchannel,
(b) means for introducing particles into said microchannel,
(c) means for detecting a label on a particle passed through said microchannel,
(d) means for removing a labeled particle from unlabeled particles,
which device is characterized in that the means (c) and (d) are designed in such a way that they provide for repeating at least once the detection/removal procedure.
